(19) United States
(12) Patent Application Publication
(10) Pub. No.: US 2019/0090774 A1
(43) Pub. Date: Mar. 28, 2019

(54) SYSTEM AND METHOD FOR LOCALIZATION OF ORIGINS OF CARDIAC ARRHYTHMIA USING ELECTROCARDIOGRAPHY AND NEURAL NETWORKS

(71) Applicant: Regents of the University of Minnesota, Minneapolis, MN (US)

(72) Inventors: Ting Yang, Minneapolis, MN (US); Bin He, Minneapolis, MN (US)

(21) Appl. No.: 16/142,213

(22) Filed: Sep. 26, 2018

Related U.S. Application Data
(60) Provisional application No. 62/563,862, filed on Sep. 27, 2017.

(51) Int. Cl. A61B 5/0468; A61B 5/044; A61B 5/0428; A61B 6/03; A61B 5/0472; A61B 18/00; G06N 3/08; G16H 50/20

(52) U.S. Cl. CPC A61B 5/0468; A61B 5/044; A61B 5/04286; A61B 6/032; A61B 5/7264; A61B 18/00; G06N 3/08; G16H 50/20; A61B 5/0472

(57) ABSTRACT

Disclosed are methods and systems for localizing where in a heart an arrhythmia originates. Electrical data may be recorded using an electrocardiography device, the electrical data corresponding to electrical activity in the heart of a subject. The electrical data (or portions or representations thereof) may be fed to one or more convolutional neural networks. The one or more neural networks may provide an identification of a segment of the heart at which an arrhythmia originates, and whether the arrhythmia has an epicardial or endocardial focus. Arrhythmias may localized and classified non-invasively using only data acquired using, for example, a 12-lead ECG.

### CROSS-REFERENCE TO RELATED APPLICATIONS

This application claims the benefit of U.S. Provisional Patent Application Ser. No. 62/563,862 filed on Sep. 27, 2017 and entitled “SYSTEM AND METHOD FOR LOCALIZATION OF ORIGINS OF CARDIAC ARRHYTHMIA USING ELECTROCARDIOGRAPHY AND NEURAL NETWORKS.”

### STATEMENT REGARDING FEDERALLY SPONSORED RESEARCH

N/A

### FIELD OF THE INVENTION

This document relates to systems and methods for localizing activity in cardiac tissue using data acquired non-invasively via electrocardiography.

### BACKGROUND

The 12-lead electrocardiogram (ECG), which can be recorded relatively rapidly is widely used in diagnosing heart conditions. In an ECG, a normal heart beat produces four entities: a P wave, a QRS complex, a T wave, and a U wave. These four entities represent different electrophysiological stages of the heart: atrial depolarization, ventricular depolarization, ventricular repolarization, and papillary muscle repolarization. Among them, the QRS complex is of the most interest to cardiac electrophysiologists since a majority of ventricular arrhythmias are reflected by changes in morphology on the QRS complex.

A premature ventricular contraction (PVC) is a type of ectopic beat, in which the heart beat is initiated by an ectopic pacemaker in the ventricles. It is one of the most common ventricular arrhythmias and its prevalence is associated with many factors. The prevalence of PVC is estimated to be greater than 6%, based on a large cross-sectional study in which 15,792 adults (aged 45-65 years) where administered a 2-minute ECG. If not treated in time, the condition of PVC may degenerate into other ventricular arrhythmias such as ventricular tachycardia (VT) and ventricular fibrillation (VF) and finally lead to sudden cardiac death.

Radiofrequency catheter ablation is an invasive procedure that aims to treat patients by delivering energy to sections of the heart that are prone to producing arrhythmias in order to terminate the arrhythmias. To localize the site of ablation, pace-mapping is a predominant technique used in clinical settings. If the morphology of the QRS complex by pacing at a site matches well with the VT or PVC observed on the 12-lead ECG, this site is considered to be a potential ablation site. Pace-mapping is done by stimulating at different endocardial sites, so it is invasive. Also, the better the localization to be achieved (i.e., the lower the error in location of a source of an arrhythmia), the more extensive the invasive procedure (requiring, e.g., an increased number of electrodes). In addition, errors in locating a source of an arrhythmia can lead to damage to healthy tissue surrounding the actual source.

Researchers have been investigating features of the QRS complex obtained from the 12-lead ECG to help identify ablation targets for PVC and VT. Some typical characteristics of the QRS complex used for localization are QRS width, QRS axis, QRS patterns (qR, QS, RsR′ and so on), R wave amplitude and concordance. Some characteristics of the QRS complex can be calculated automatically, but for other features that involve pattern recognition, characterizing the QRS complex requires expertise obtained from many years of training, and even with training, is ultimately a subjective process.

What is needed is an objective, non-invasive approach to localizing where an arrhythmia originates in the heart.

### SUMMARY OF THE PRESENT DISCLOSURE

In one or more example embodiments of the present disclosure, a method for localizing where in a heart an arrhythmia originates may comprise: acquiring electrical data recorded using an electrocardiography device, the electrical data corresponding to electrical activity in the heart of a subject; feeding the electrical data to one or more neural networks; and receiving from the one or more neural networks an identification of a segment of the heart at which an arrhythmia originates.

In various implementations: the identification of the segment may comprise generating and displaying an image visually depicting the segment of the heart, relative to other segments, in which the arrhythmia originates; the arrhythmia may be localized non-invasively; the electrical data may be the only physiological data recorded from the heart of the subject used for identifying the segment at which the arrhythmia originates; the CT or MRI data of the subject's torso and heart may also be recorded; the electrocardiography device with which the electrical data is acquired may be a 12-lead ECG; electrical data from the 12-lead ECG may be the only physiological data input into the one or more convolutional neural networks (CNNs) to receive an identification of the segment of the heart at which the arrhythmia originates; the one or more neural networks may include one or more CNNs; the one or more CNNs may include a segment CNN that is configured to receive, as input, full time courses of an electrocardiogram, and provide, as output, the segment of the heart at which the arrhythmia originates; the segment CNN may include two hidden layers and two pooling layers; the CNN may be an epi-endo (epicardial-endocardial) CNN that is configured to receive, as input, a portion of a QRS complex acquired from an electrocardiogram, and provide, as output, an identification of whether the arrhythmia has an epicardial focus or an endocardial focus; the portion of the QRS complex may be the first half of the QRS complex; the one or more neural networks may include: a segment CNN configured to provide the identification of the segment; and an epi-endo CNN configured to identify whether the arrhythmia has an epicardial focus or an endocardial focus; the segment CNN may receive as input a full time course of an electrocardiogram, and the epi-endo CNN may receive as input a portion of a QRS complex from the electrocardiogram; the neural networks may be CNNs trained on simulation data; the method may further comprise using a realistic anisotropic ventricle computer model of the heart to generate QRS complexes of 12-lead ECG from pacing at all possible ventricular locations; the method may further comprise treating the segment of the heart at which the arrhythmia originates; the segment may be treated via ablation.

In one configuration, a method is provided for identifying an arrhythmia origination location in a heart of a subject. The method includes acquiring electrical data recorded using an electrocardiography device, where the electrical data corresponds to electrical activity in the heart of the subject. The electrical data may be provided to one or more neural networks and identification of a segment of the heart at which an arrhythmia originates may be received from the one or more neural networks.

In one configuration, a system is provided for identifying an arrhythmia origination location in a heart of a subject. The system includes an electrocardiography device to record electrical data corresponding to electrical activity in the heart. The system may also include a processor and memory having instructions that, when executed by the processor, are to: use the electrocardiography device to record electrical activity in the heart of the subject, provide the electrical data to one or more neural networks, and receive from the one or more neural networks an identification of a segment of the heart at which an arrhythmia originates.

In one or more example embodiments, a system for localizing where in a heart an arrhythmia originates may comprise: an electrocardiography device configured to record electrical data corresponding to electrical activity in the heart; a processor and memory having instructions that, when executed by the processor, are configured to: use the electrocardiography device to record electrical activity in the heart of a subject; feed the electrical data to one or more neural networks; and receive from the one or more neural networks an identification of a segment of the heart at which an arrhythmia originates.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the invention.

### BRIEF DESCRIPTION OF THE DRAWINGS

The following drawings are provided to help illustrate various features of example embodiments of the disclosure, and are not intended to limit the scope of the disclosure or exclude alternative implementations.

FIG. 1A is a discretized image of the heart illustrating example segmentations of ventricles in the heart.

FIG. 1B is a chart showing one configuration for a right ventricle segmentation.

FIG. 1C is a chart showing one configuration for a left ventricle segmentation.

FIG. 1D is an image of the heart showing endo-surfaces of both ventricles and also the epicardium.

FIG. 2A provides a training and testing study diagram corresponding to an example Segment CNN (convolutional neural network). All cardiac dipoles are multiplied by the leadfield of 12-lead ECG to get the time courses of electric potentials. Then different levels of Gaussian white noise were added to the potentials. 90% of the QRS complexes from 12-lead ECG were selected as the input to Segment CNN for training procedure. The other 10% were used for testing. The output of Segment CNN is a probability distribution among 25 example segments, the segment with the maximum probability is considered as the output segment.

FIG. 2B provides a training and testing study diagram corresponding to an example Epi-Endo CNN. Cardiac dipoles located at epicardium and endocardium were selected and multiplied by the leadfield of 12-lead ECG. Different levels of Gaussian white noise were added and the first half of QRS complexes served as the input for Epi-Endo CNN. 90% of the data were for training and the other 10% were for testing. The output is a probability distribution among two output neurons, either neuron 1 (Epi) or neuron 2 (Endo) would have a larger probability.

FIG. 3A provides an example precision matrix of Segment CNN trained and tested on a first of 3 different SNR signals: specifically, 20 dB. Each column represents the precision of this segment over all the other segments. All the blocks with precision higher than 5% are labeled.

FIG. 3B provides an example precision matrix of Segment CNN trained and tested on a second of 3 different SNR signals: specifically, 10 dB. Each column represents the precision of this segment over all the other segments. All the blocks with precision higher than 5% are labeled.

FIG. 3C provides an example precision matrix of Segment CNN trained and tested on a third of 3 different SNR signals: specifically, 5 dB. Each column represents the precision of this segment over all the other segments. All the blocks with precision higher than 5% are labeled.

FIG. 4 provides simulation results for an example CNN. The top two lines are the average accuracy of Segment CNN and Epi-Endo CNN trained and tested on 3 different SNR signals. The bottom bars are the average localization errors when applying Segment CNN and Epi-Endo CNN together to data with different SNRs.

FIG. 5 provides average localization errors of a variety of heart registration errors.

FIG. 6 provides results corresponding to nine PVC patients according to one or more example embodiments. The right y-axis represents the accuracy and average precision rate of the trained Segment CNN and Epi-Endo CNN. And the left y-axis shows the average localization error of 10 PVCs from each of 9 patients. On the bottom, x-axis is the noise level estimated from 12-lead ECGs and is also the noise level we trained and tested the CNNs by adding Gaussian white noise.

FIG. 7 depicts an example system that could be used to implement example embodiments, or portions thereof, of the present disclosure.

### DETAILED DESCRIPTION OF THE PRESENT DISCLOSURE

Example embodiments of the present disclosure classify and localize origins of cardiac arrhythmias throughout the ventricles by applying neural networks to 12-lead ECG. The ventricles may be divided into segments, such as 25 segments, based on standard myocardial segmentation of the left ventricle and expanded to the right ventricle. A realistic anisotropic ventricle computer model may be used to generate QRS complexes of ECG leads, such as 12-lead ECG, from pacing at all possible ventricular locations. The time course of QRS complexes generated may then be fed to one or more convolutional neural networks (CNNs) for classification and localization purposes.

Epicardial mapping and ablation have expanded considerably in the past few years. It was reported that 13% to 17% VT ablation procedures were epicardial mapping or ablation. On ECG, epicardial VT is reflected as a slow onset of the QRS because the initial part of the wave front progresses slowly until it reaches the Purkinje system at the subendocardium. The intracardiac delay of electrical conduction produces a slurred initial part of the QRS complex (pseudo Δ wave). So the initial part (first half) of the QRS is critical for the detection of an epicardial or endocardial focus. In the disclosed study, which illustrates concepts and features of example embodiments of the present disclosure, we use the first half of QRS complexes generated by the ventricle computer model as input to the CNN to classify between an epicardial ectopic beat and an endocardial ectopic beat.

One example implementation includes using two neural networks: Segment CNN with 25 classifications and Epi-Endo CNN with 2 classifications. The localization of origins of PVC is a function of probability distribution outputs of the two CNNs and the center of gravity of each segments in the ventricle model. The discussed example approach was applied to real 12-lead ECG collected from 9 PVC patients who underwent ablation treatment.

Prior studies that attempted to localize the origin of PVC from the 12-lead ECG used myocardial activation imaging technique based on an equivalent double layer model to localize the PVC origin, although positions of 12-lead ECG were unknown, and the accuracy of results was thus reduced. Also, a quantitative measurement of localization error was lacking in the prior studies. In one example study being presented as an illustration of example implementations of the disclosure, electrodes' positions were digitized and the average localization error was presented by calculating the spatial distance between the CNN predicted sites of origin of PVC and successful ablation sites recorded from EP study in the patient. Example approaches disclosed are a step forward from prior approaches and model-based noninvasive identification of ablation targets. For a more accurate estimation of cardiac activation sequence to localize origins of PVC or VT, a noninvasive 3-dimensional cardiac electrical imaging technique was developed based on the body surface potential mapping. It was evaluated under a variety of animal studies, in pathological hearts, and applied to detect high-frequency drivers of atrial fibrillation.

Method: For one study being presented as an illustration of example implementations, it was assumed that PVCs are generated by focal sources, and if a CNN is trained with all the possible 12-lead ECGs resulting from a single-site pacing covering the ventricular volume with a certain level of noise, the CNN will be able to identify which segment the origin of PVC lies in and whether it is an epicardial or endocardial source given a set of 12-lead ECGs. And depending on the probability distribution of CNN output, we could also give an estimation of source location based on the classification information.

Realistic Anisotropic Ventricle Computer Model: The ventricular excitation was simulated by a cellular automaton heart model. Heart geometry was constructed from CT images of a human subject and discretized into around 38,000 cellular units with a side length of 1.5 mm (see B. He et al, “Noninvasive imaging of cardiac transmembrane potentials within three-dimensional myocardium by means of a realistic geometry anisotropic heart model,” IEEE Trans. Biomed. Eng., vol. 50, no. 10, pp. 1190-1202, 2003). Then a generalized ventricle conduction anisotropy was incorporated into the model. The myocardial fiber orientations rotated counterclockwise over 120° from the outermost layer (epicardium, −60°) to the innermost layer (endocardium, +60°) with equivalent increment between the consecutive layers. The conduction velocity along the fiber was 0.6 m/s, and 0.2 m/s transverse to the fiber, respectively. Also, the longitude intracellular conductivity was set as 0.3 S/m and the transverse intracellular conductivity was 0.075 S/m, respectively. The action potential and the vector of local fiber orientation were set individually over all the ventricular cellular units. The equivalent current-dipole density of each unit was computed as the product of the myocardial conductivity tensor and the spatial gradient of instantaneous transmembrane potential. Each dipole has three orthogonal components. Finally, cellular units were further grouped into 3,887 dipoles according to their segment number. The time resolution of electric potentials is 1 millisecond. (Background for this ventricle current-dipole model in the context of a simulation study of a 3-dimensional cardiac imaging technique can be found at: Z. Liu et al, “Noninvasive reconstruction of three-dimensional ventricular activation sequence from the inverse solution of distributed equivalent current density,” IEEE Trans. Med. Imaging, vol. 25, no. 10, pp. 1307-1318, 2006; L. Yu et al, “Temporal sparse promoting three dimensional imaging of cardiac activation,” IEEE Trans. Med. Imaging, vol. 34, no. 11, pp. 2309-2319, 2015.)

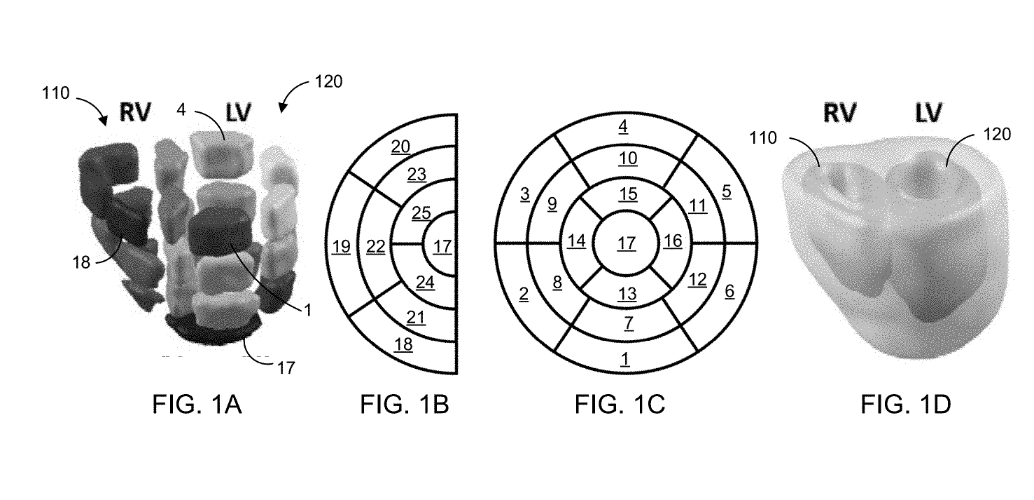

Referring to FIGS. 1A-1D, one configuration for segmentation of the whole ventricle and extraction of epicardium and endocardium is shown. In some configurations, segmentation may follow the American Heart Association (AHA) standardized myocardial segmentation. In the ventricle current-dipole model, which consists of 3,887 cardiac dipoles, the left ventricle 120 was segmented according to AHA standardized myocardial segmentation into 17 segments, and the right ventricle 110 was segmented in a similar way as the left ventricle into 8 segments. Thus, the whole ventricle was classified into 25 segments in total. The position and the number of each segment are shown in FIG. 1B for the right ventricle 110, and FIG. 1C for the left ventricle 120. Left ventricle 120 labels may include: basal anterior 1, basal anteroseptal 2, basal inferoseptal 3, basal inferior 4, basal inferolateral 5, basal anterolateral 6, mid anterior 7, mid anteroseptal 8, mid inferoseptal 9, mid inferior 10, mid inferolateral 11, mid anterolateral 12, apical anterior 13, apical septal 14, apical inferior 15, apical lateral 16, and apex 17. Right ventricle 110 labels may include: basal anterior 18, basal lateral 19, basal inferior 20, mid anterior 21, mid lateral 22, mid inferior 23, apical anterior 24, and apical inferior 25. The number of cardiac dipoles each segment includes is listed in Table A. FIG. 1A is a visualization of 25 segments. FIG. 1D is a image of the heart showing the epicardium and endocardium. In one example model, the dipoles lying on epicardium and endocardium were identified and labeled as one and two, respectively, and all other dipoles were assigned three as transmural dipoles. Both the left side and the right side of the septum are considered as endocardium.

TABLE A
Example Number of Dipoles per Segment

| | Number | | Number |
|---|---|---|---|
| Left Ventricle Label | of Dipoles | Right Ventricle Label | of Dipoles |
| basal anterior 1 | 232 | basal anterior 18 | 101 |
| basal anteroseptal 2 | 150 | basal lateral 19 | 100 |
| basal inferoseptal 3 | 179 | basal inferior 20 | 159 |
| basal inferior 4 | 192 | mid anterior 21 | 112 |
| basal inferolateral 5 | 171 | mid lateral 22 | 110 |
| basal anterolateral 6 | 155 | mid inferior 23 | 125 |
| mid anterior 7 | 170 | apical anterior 24 | 74 |
| mid anteroseptal 8 | 139 | apical inferior 25 | 102 |
| mid inferoseptal 9 | 165 | | |
| mid inferior 10 | 164 | | |
| mid inferolateral 11 | 146 | | |
| mid anterolateral 12 | 128 | | |
| apical anterior 13 | 193 | | |
| apical septal 14 | 195 | | |
| apical inferior 15 | 179 | | |
| apical lateral 16 | 183 | | |
| apex 17 | 263 | | |

12-lead ECG for Simulation: The torso and lungs were segmented from high-resolution male torso MRI images (ViP V2.0 IT'IS Foundation, Zürich, Switzerland). Then the boundary element model of heart-torso was built using a commercial software (Curry 6.0, Neuraoscan, North Carolina, USA). 208 body surface electrodes were placed on the front and back of body surface. By multiplying the current-dipoles with the leadfield matrix between each dipole and each electrode, we could get body surface potentials. Among these signals, nine were chosen to generate 12-lead ECG. Three electrodes were selected to represent the electrodes placed on left leg, left arm, and right arm, respectively. So lead I, II, III and lead aVL, aVR and aVF could be derived from the signals of these electrodes. Other six electrodes were selected as V1 to V6 according to their anatomical locations.

Training and Testing of Convolutional Neural Network: Convolutional neural network (CNN) is a type of deep, feed-forward artificial neural network. As part of an example implementation, we used a deep learning toolbox in MATLAB developed by Rasmus Berg Palm to setup, train and test CNN. It uses a sigmoid function as the activation function for feed forward propagation and a gradient descent projection method as the back propagation algorithm. Convolution and pooling were done in 2-dimension (2-D). The stride, namely the number of unit that convolutional kernel shifts each time, is equal to 1. There is no padding in convolutional layers. The 2-D convolutional kernel has an equal size along both directions. To ensure the applicability of the convolutional kernel, the input should also be in square shape. Thus, we added 4 more leads derived from three limb leads (LL for left leg, RA for right arm, LA for left arm): LL−RA−LA; LA−RA−LL; RA−LA−LL; (LL+RA+LA)/3. The last one corresponds to the Wilson central terminal. Along the time dimension, we down-sampled the time courses of QRS (or first half of QRS) to 16 time points before adding noise in simulation. Therefore, each input set of ECGs is a 16×16 square matrix. This size results in the fast response of both CNNs.

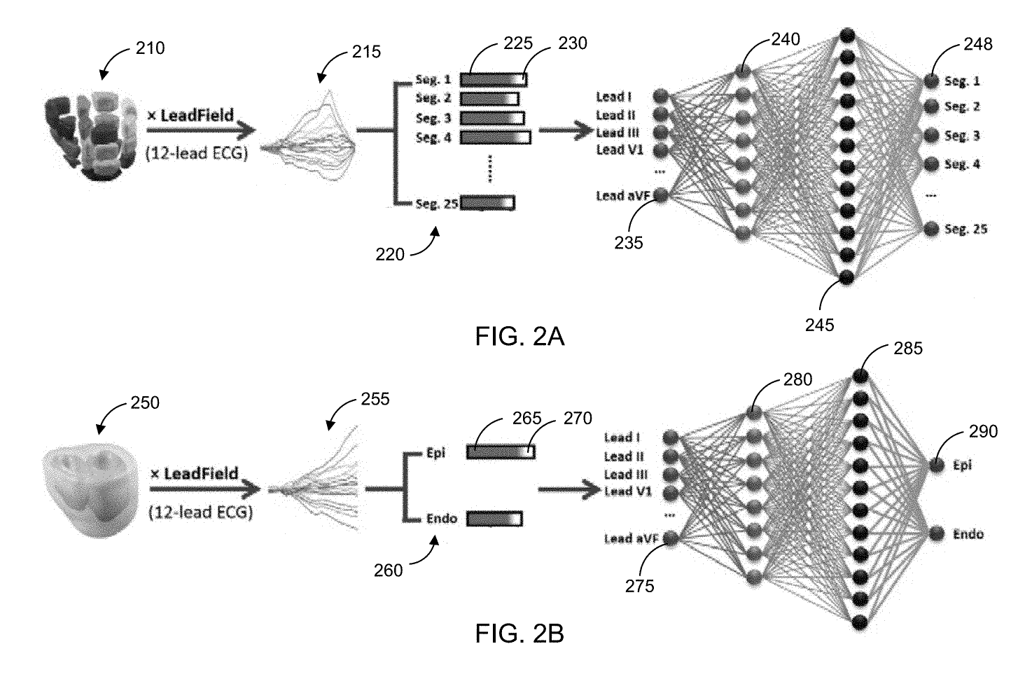

In order to localize the origins of PVCs, we utilized the classification information from two types of CNN: Segment CNN and Epi-Endo CNN. The study diagram of this section is shown in FIGS. 2A, and 2B respectively.

Referring to FIG. 2A, one configuration for a segment CNN is shown. A segmented model 210, which may be a model as depicted in FIGS. 1A-1D, is used as an input. The cardiac dipole components resulted from pacing may be multiplied by the lead field matrix to generate body surface electrical potentials. Thus, we would have 3,887 sets of 12-lead ECGs corresponding to pacing at 3,887 locations in the ventricle model. QRS complexes 215 were extracted from each lead. A certain level of Gaussian white noise (20 dB, 10 dB or 5 dB) was added to all the leads ten times to generate noise contaminated 12-lead QRS complexes. Segments 220 may be trained and tested separately. Adding a certain level of noise ten times is for increasing the robustness of Segment CNN to this level of noise since we would have ample training samples 225 representing different noise variations. In total, our data pool had 38,870 sets of noise-contaminated 12-lead QRS complexes. Testing data 230 made up 10% of the whole data, which means 3,887 sets were the testing data. In order to test the CNN without segment bias, within each segment, 155 (≈3,887/25) sets of 12-lead QRS complexes were randomly selected each time for testing. The remaining 90% data were used for training procedure. The Segment CNN consisted of 6 layers: Input layer 235 (A set of 12-lead QRS complexes), a first Hidden layer 240 (convolutional layer with a kernel size of 5), a Pooling layer 1 (not shown, sampling scale of 1), a second Hidden layer 245 (convolutional layer with a kernel size of 3), a second Pooling layer (not shown, sampling scale of 2), and Output layer 248 (25 neurons for 25 segments). The batch size was 23, alpha was 1 and the number of epochs was 10. After a ten-fold cross-validation, the accuracy and precision rate of each segment of Segment CNN were calculated.

Referring to FIG. 2B, one configuration for an Epi-Endo CNN is shown. Similar to Segment CNN, 1,709 dipole sources located on epicardium and endocardium 250 were identified and were used to generate single-site pacing 12-lead ECGs. QRS complexes 255 were extracted and the first half of 12-lead QRS complexes was used. Gaussian white noise of a specific SNR was added 10 times. Thus for Epi-Endo CNN, we had 17,090 sets of first half of QRS complexes from 12 leads. Epicardium and endocardium surfaces 260 may be analyzed separately. Testing data 270 made up 10% of this data, which means 855 (≈1,709/2) sets were randomly selected from each category. The remaining 90% data were used for training data 265. The Epi-Endo CNN consisted of 6 layers: Input layer 275 (first half of QRS complexes from 12-lead ECGs), a first Hidden layer 280 (convolutional layer with a kernel size of 5), a first Pooling layer (not shown, sampling scale of 2), a second Hidden layer 285 (convolutional layer with a kernel size of 3), a second Pooling layer (not shown, sampling scale of 2), and Output layer 290 (2 neurons corresponding to epicardium and endocardium). Parameters were set as follows: batch size=25, alpha=3 and number of epochs=10. After a ten-fold cross-validation, the accuracy and precision rate of Epi or Endo were calculated.

Localization of Origins of PVCs: After training procedures, we tested the localization performance of these two CNNs by feeding a new set of data, which included 12-lead ECGs with a certain SNR resulting from pacing at all possible dipole locations. For each set of 12-lead ECGs, Segment CNN would assign a probability to each segment and Epi-Endo CNN would tell how possible it was from epicardium or endocardium. Based on these two probability distributions, the estimation of source location was calculated as following:

$$S = {\sum }_{i = 1}^{N} {P}_{i} \times ( {\sum }_{j = 1}^{2} {P}_{j} \times {CoG}_{ij} ) \qquad ( 1 )$$

In Eq. (1), S is source location, Pᵢ is the normalized probability of ith segment and its adjacent segments, the output of Segment CNN; N is the number of adjacent segments each segment has based on the ventricle segmentation in FIGS. 1A-D; Pⱼ is the probability of Epi or Endo, the output of Epi-Endo CNN; and j is 1 for Epi; 2 for Endo. CoGᵢⱼ is the center of gravity of Epi or Endo dipole sources in ith segment.

The segment with the maximum probability from Segment CNN was considered as the output segment. Its adjacent segments (sharing boundaries with the output segment) were determined based on the ventricle segmentation in FIGS. 1A-D. The endocardial center of gravity and epicardial center of gravity in each segment were calculated. By multiplying spatial locations of epicardial and endocardial centers of gravity with the probabilities of epicardium and endocardium from Epi-Endo CNN, we obtained epi-endo-informed centers of gravity of the output segment and its adjacent segments. This process corresponds to the multiplication inside the parentheses in Eq. (1). Since some dipole sources locate near the boundary of two segments, it was probable that Segment CNN would assign it to its neighboring segment. This is why we took the adjacent segments of an output segment into consideration to decrease the localization error (LE). And it was done by first normalizing the output probabilities of Segment CNN only among the output segment and its adjacent segments, and then multiplying the normalized probability with each epi-endo-informed center of gravity. Adding all up, we estimated the source location.

Application to PVC Patients: The present example approach was tested in 9 PVC patients. None of the testing patients have undergone ablation procedure before and none of them have structural heart disease. For each patient, we collected CT images of heart, lungs and torso. The heart and torso CT images were coupled based on some important cardiac anatomical landmarks, such as the apex of the heart. Then the manual segmentation and building a boundary element model were done in a commercial software (Curry 6.0, Neuroscan). After the segmentation of patient's heart, we registered our generic ventricle model with the patient's heart based on anatomical landmarks such as the apex and the septum, and minimized the sum of distances between the two. Prior to the EP study, 208 Ag—AgCl carbon electrodes were placed on the front and back of body surface to record body surface electrical potentials with 2 kHz sampling rate for about 10-30 minutes and positions of electrodes were digitized (Fastrak, Polhemus Inc., Colchester, Vt., USA). During the recording, all of the patients have spontaneous PVCs recorded and 2 of them also have non-sustained VT detected. Nine electrodes were selected to form 12-lead ECGs later based on their anatomical positions. By using Curry 6.0, the leadfield between these nine electrodes and our registered generic ventricle model was generated. A personalized leadfield helps the training of the two convolutional neural networks to present a unique relationship between the cardiac activities from the generic ventricle model and the body surface ECGs of a patient, and the personalized leadfield was built upon CT images of the heart and torso. After the EP study, we also collected CARTO files, which contained spatial locations of successful ablation sites. And CARTO data were registered with the patient's heart based on the landmarks of the geometry of ventricles recorded by the CARTO system. The successful ablation sites are considered as the origins of PVCs in this study.

QRS complexes of 10 PVCs were exported from body surface potential recordings of each patient. Body surface recordings were first filtered by a bandpass filter from 1 Hz to 30 Hz. After filtering, the QRS and the first half of QRS were down-sampled to 16 time points respectively to construct input for Segment CNN and Epi-Endo CNN. To estimate the SNR, signals of the same length as QRS complexes of PVCs between heart beats were also exported and were considered as noise. Average SNR is defined as the average of SNR of each channel. Then we trained and tested two CNNs using simulated 12-lead ECGs with the average SNR between QRS complexes and Gaussian white noise. Training and testing details are described in Section D. The only difference is that the leadfield used to generate 12-lead ECGs here is specific to each patient. Finally, 12-lead ECGs of 10 PVCs were fed into two trained CNNs to determine source locations.

Statistical Analysis: The accuracy of Segment CNN and Epi-Endo CNN were presented as mean±SD (i.e., standard deviation). The localization error was defined as the spatial distance between the estimated source location and the location of the dipole source in the generic ventricle model that generates the 12-lead ECGs by pacing in simulation, and was defined as the spatial distance between the estimated source location and successful ablation sites in patients' CARTO data.

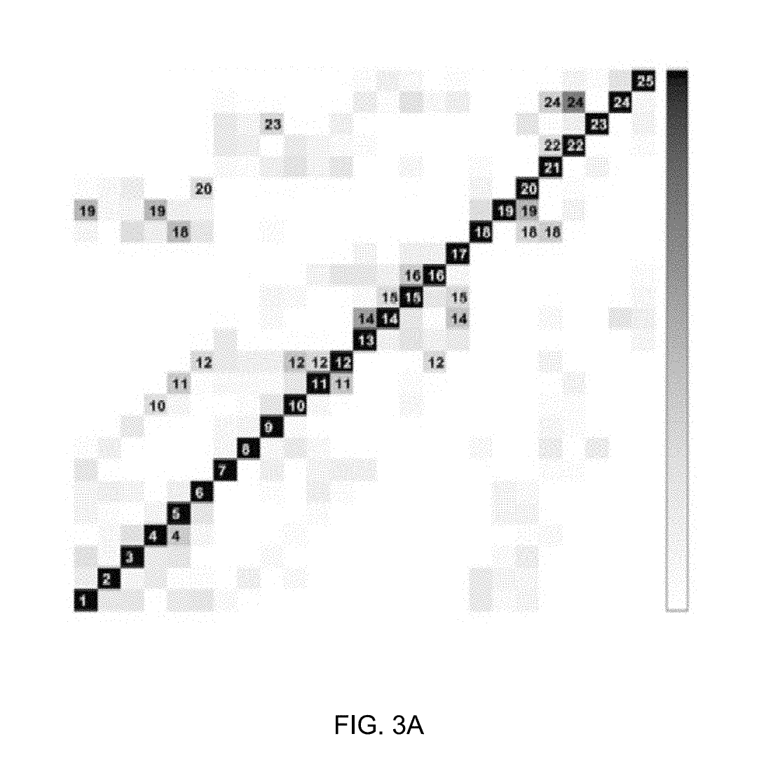

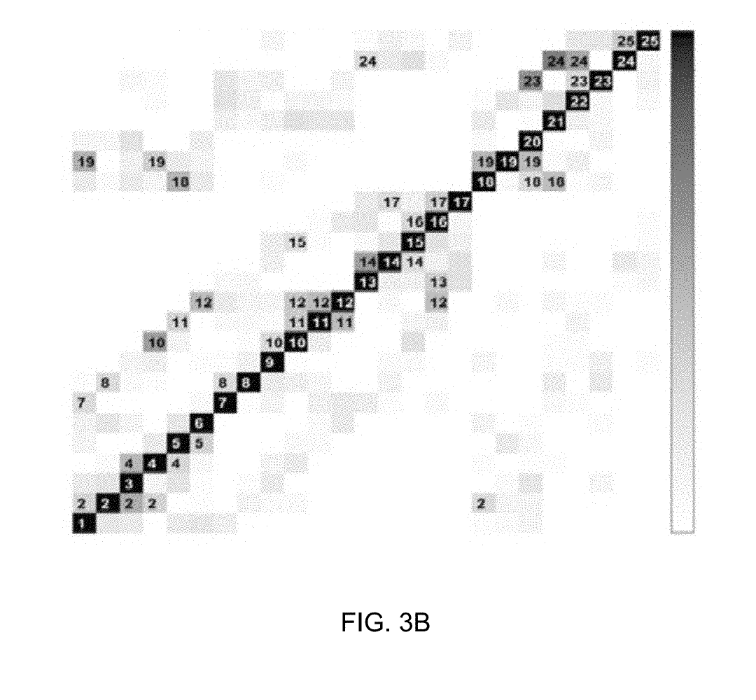

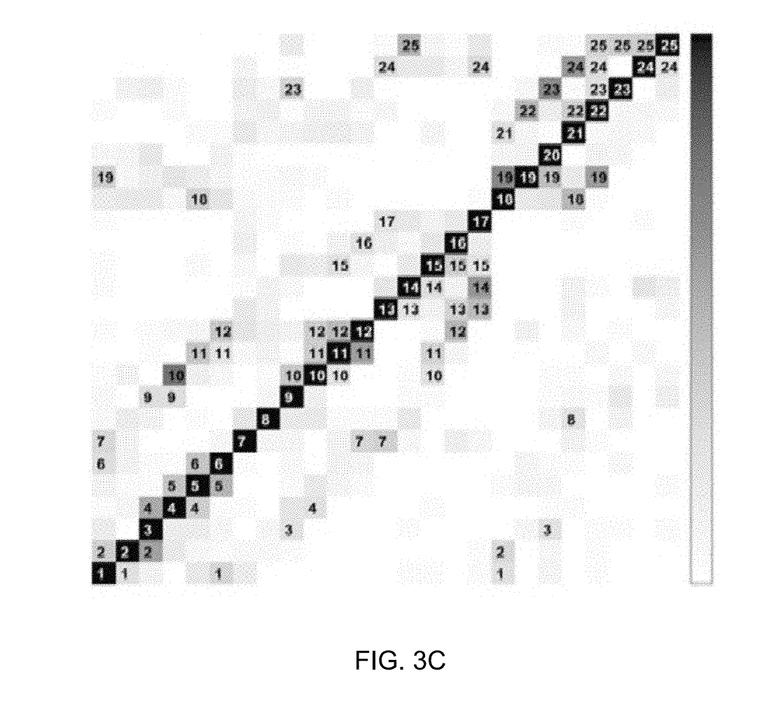

Results: Table 1 shows the accuracy and precision of individual segments when Segment CNN was trained and tested on three different Gaussian white noise levels. Accuracy is defined as the percentage of dipole sources being correctly classified within each segment. Precision is defined as when Segment CNN predicts it is in one segment, how often it is correct. FIGS. 3A-3B show the precision matrices of Segment CNN. Each column is the precision vector for this segment. When the probability of Segment CNN assigning this segment to other segments is higher than 5%, it is labeled in black in the figure. For example, when trained and tested on 20 dB (FIG. 3A), for segment 4, Segment CNN would have a relative higher probability of assigning segment 19 and segment 10 to segment 4 even though the precision for segment 4 is 74.1% from Table 1. Table 2 is the precision matrices of Epi-Endo CNN trained and tested on different noise levels.

TABLE 1
Accuracy and precision rates for individual segments of Segment CNN trained and
tested on 3 different SNR signals

| | SNR = 20 dB | | SNR = 10 dB | | SNR = 5 dB | |
|---|---|---|---|---|---|---|
| Unit (%) | Accuracy | Precision | Accuracy | Precision | Accuracy | Precision |
| 1 | 75.6 | 72.7 | 77.0 | 65.1 | 63.6 | 53.9 |
| 2 | 80.5 | 85.2 | 61.9 | 78.4 | 54.5 | 70.7 |
| 3 | 78.4 | 73.9 | 72.0 | 61.1 | 65.7 | 48.2 |
| 4 | 79.6 | 74.1 | 71.6 | 64.0 | 56.3 | 51.2 |
| 5 | 85.7 | 62.3 | 80.0 | 61.6 | 60.3 | 46.8 |
| 6 | 83.7 | 72.4 | 80.6 | 68.8 | 69.0 | 52.3 |
| 7 | 84.4 | 79.6 | 77.4 | 74.4 | 59.0 | 58.3 |
| 8 | 81.5 | 85.5 | 71.2 | 82.3 | 60.7 | 74.0 |
| 9 | 89.0 | 74.6 | 83.2 | 66.5 | 71.2 | 49.3 |
| 10 | 82.5 | 73.5 | 68.4 | 63.1 | 46.8 | 56.7 |
| 11 | 72.5 | 79.7 | 67.3 | 75.3 | 49.9 | 63.1 |
| 12 | 62.5 | 77.5 | 52.0 | 73.9 | 41.7 | 62.6 |
| 13 | 81.6 | 75.4 | 77.4 | 71.6 | 58.6 | 69.8 |
| 14 | 66.5 | 86.1 | 65.8 | 75.5 | 53.3 | 61.8 |
| 15 | 79.6 | 72.7 | 75.9 | 70.2 | 60.5 | 60.3 |
| 16 | 77.5 | 83.2 | 83.5 | 66.6 | 77.3 | 60.8 |
| 17 | 92.6 | 78.3 | 80.4 | 83.7 | 80.2 | 54.2 |
| 18 | 58.4 | 84.0 | 52.5 | 75.7 | 40.4 | 62.5 |
| 19 | 54.8 | 91.0 | 48.5 | 85.7 | 25.5 | 71.4 |
| 20 | 87.1 | 70.8 | 84.4 | 61.7 | 81.7 | 52.3 |
| 21 | 81.7 | 70.4 | 74.8 | 62.6 | 71.6 | 47.1 |
| 22 | 79.5 | 75.1 | 72.7 | 69.2 | 63.0 | 58.8 |
| 23 | 75.4 | 93.8 | 62.6 | 80.9 | 42.8 | 77.3 |
| 24 | 62.6 | 91.1 | 56.6 | 88.7 | 42.9 | 82.5 |
| 25 | 89.3 | 89.3 | 80.2 | 89.7 | 57.7 | 79.0 |

TABLE 2
The Precision Matrix of Epi-Endo CNN trained and tested on 3
different SNR signals

| | | |
|---|---|---|
| SNR = | SNR = | SNR = |
| 20 dB | 10 dB | 5 dB |

| Unit (%) | Epi | Endo | Epi | Endo | Epi | Endo |
|---|---|---|---|---|---|---|
| Epi | 85.56 | 9.94 | 72.79 | 18.64 | 61.36 | 21.79 |
| Endo | 14.44 | 90.06 | 27.21 | 81.36 | 38.64 | 78.21 |

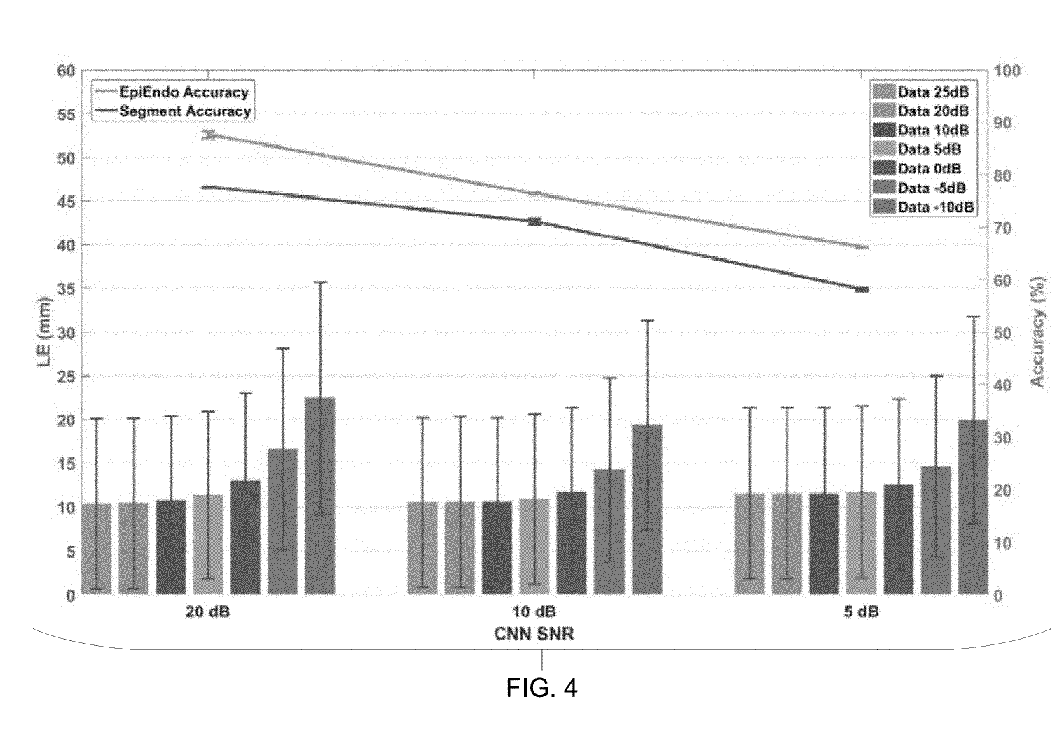

When the localization performance of two example CNNs was tested, they were tested by feeding them 3,887 new sets of 12-lead ECGs by pacing at a single site with a variety of noise levels (25 dB, 20 dB, 10 dB, 5 dB, 0 dB, −5 dB and −10 dB). FIG. 4 presents the LEs along with average accuracies of Segment CNN and Epi-Endo CNN. The left part of the figure is the average LE when Segment CNN and Epi-Endo CNN were tested on 12-lead ECGs from all possible pacing sites in the ventricle model with different noise levels. And during the ten-fold cross validation, namely trained and tested on 20 dB 12-lead ECGs, the average accuracy of Epi-Endo CNN was 87.68% and the average accuracy of Segment CNN was 77.71%. The middle part and the right part are presented in a similar way as the left part. The exact numbers displayed on FIG. 4 are also included in Table 3.

TABLE 3
Accuracy of Segment CNN and Epi-Endo CNN (trained
and tested on 3 different SNR signals) and localization
errors of applying the CNNs under simulation

| | | | CNN SNR | | |
|---|---|---|---|---|---|
| | | | 20 dB | 10 dB | 5 dB |
| Segment Accuracy (%) | | | 77.71 ± 0.01 | 71.11 ± 0.53 | 58.18 ± 0.27 |
| EpiEndo Accuracy (%) | | | 87.68 ± 0.61 | 76.40 ± 0.07 | 66.20 ± 0.01 |
| Average | 25 | dB | 10.37 ± 9.74 | 10.56 ± 9.73 | 11.55 ± 9.74 |
| LE (mm) | 20 | dB | 10.43 ± 9.77 | 10.58 ± 9.74 | 11.55 ± 9.75 |
| of Differ- | 10 | dB | 10.76 ± 9.65 | 10.61 ± 9.64 | 11.56 ± 9.82 |
| ent Data | 5 | dB | 11.38 ± 9.51 | 10.94 ± 9.68 | 11.73 ± 9.80 |
| SNR | 0 | dB | 13.09 ± 9.97 | 11.73 ± 9.65 | 12.54 ± 9.80 |
| | −5 | dB | 16.63 ± 11.52 | 14.32 ± 10.57 | 14.66 ± 10.36 |
| | −10 | dB | 22.47 ± 13.28 | 19.36 ± 11.94 | 19.96 ± 11.85 |

Discussion: This study, intended to illustrate, without limitation, features of one or more example embodiments, shows the feasibility of utilizing convolutional neural networks to localize origins of PVCs from 12-lead ECGs. The method provides good localization errors with both simulation data as well as clinical patients' data.

Properties of example embodiments of Segment CNN and Epi-Endo CNN: The information in FIGS. 3A-C, Table 1 and Table 2 could be obtained when we train and test two CNNs on any patient-specific leadfield and under any noise level. It will guide us and give us a prior knowledge of how good two CNNs perform before we feed testing (or clinical) data to calculate the source location. From FIGS. 3A-C, we could see as SNR decreases, it is more likely for Segment CNN to assign dipole sources from other segments to one segment, which means the precision rates have dropped. The number of blocks labeled in the precision matrix increases as SNR decreases. Table 2 is the precision matrices of Epi-Endo CNN under different noise levels. The common feature of these three precision matrices is that it is more likely for Epi-Endo CNN to assign sources on endocardial surfaces as epicardial sources. From Table 1, we could know the percentage of dipole sources in each segment that are correctly detected, namely the accuracy of each segment. And also how confident we are in the detection results if one dipole source is predicted to be from some segment.

Localization Errors of CNN: From FIG. 4 and Table 3, we could summarize the following trends of the performance of two CNNs: (1) as SNR drops, the accuracies of both CNNs decrease; (2) as SNR of input data drops, the average LE increases; (3) the average LE is the result of two factors, one is the SNR that two CNNs are trained and tested, the other is the similarity between input data and tested data. The accuracy of Segment CNN of 10 dB is only 6% less than that of 20 dB. So when the SNR of input data is 10 dB and less than 10 dB, the average LEs of CNNs trained with 10 dB are all smaller than those of CNNs trained with 20 dB. This is when the similarity factor plays a dominant role. However, when the accuracy of Segment CNN drops to 58%, which is 13% less than that of 10 dB, the accuracy plays an important role in determining source locations. Because even though input data of 5 dB is more similar to CNNs trained with 5 dB, the average LE is larger than that of CNNs trained with 10 dB and input data of 5 dB. It is noted that the average LEs of CNNs trained with 5 dB are all larger than those of CNNs trained with 10 dB. Finally, when we compare the performance between CNNs trained with 20 dB and trained with 5 dB, the average LEs of input data with 0 dB and less of CNNs trained with 5 dB are smaller than those of CNNs trained with 20 dB. This is because even with a much higher training accuracy, CNNs trained with 20 dB may fail to classify 12-lead ECGs that are too unlike the training data.

Based on the above simulation results, in example embodiments, when the SNR of input data is very low, it is best to train and test CNNs with around 10 dB to minimize LEs, and when the SNR of input data is good, it is best to train and test CNNs with a noise level that is the same as the SNR of input data.

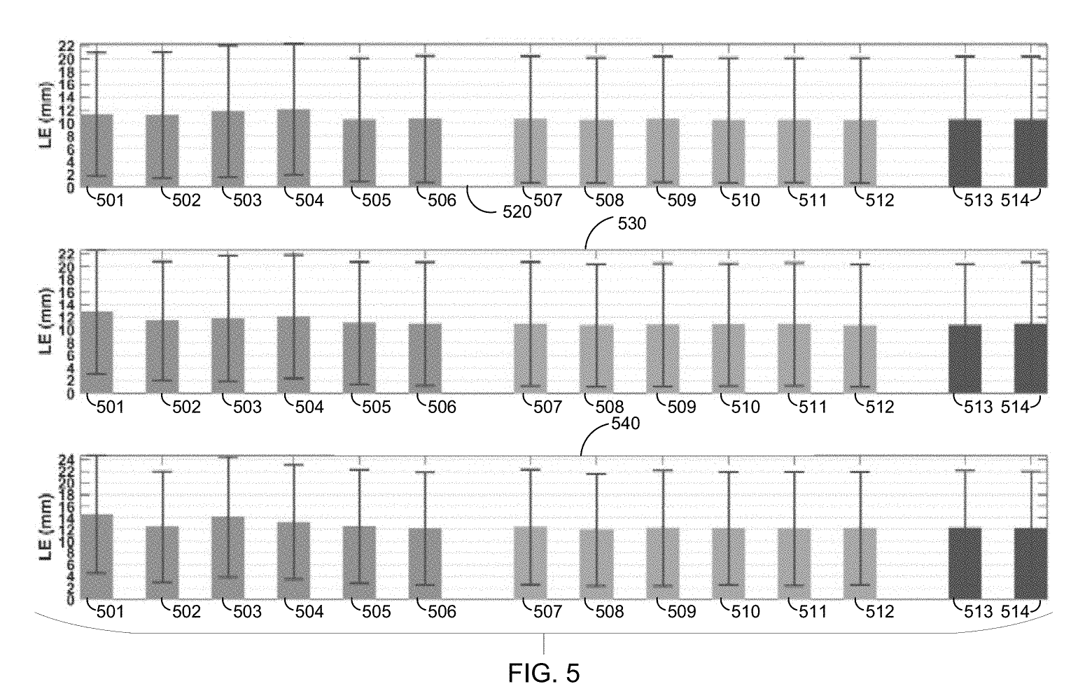

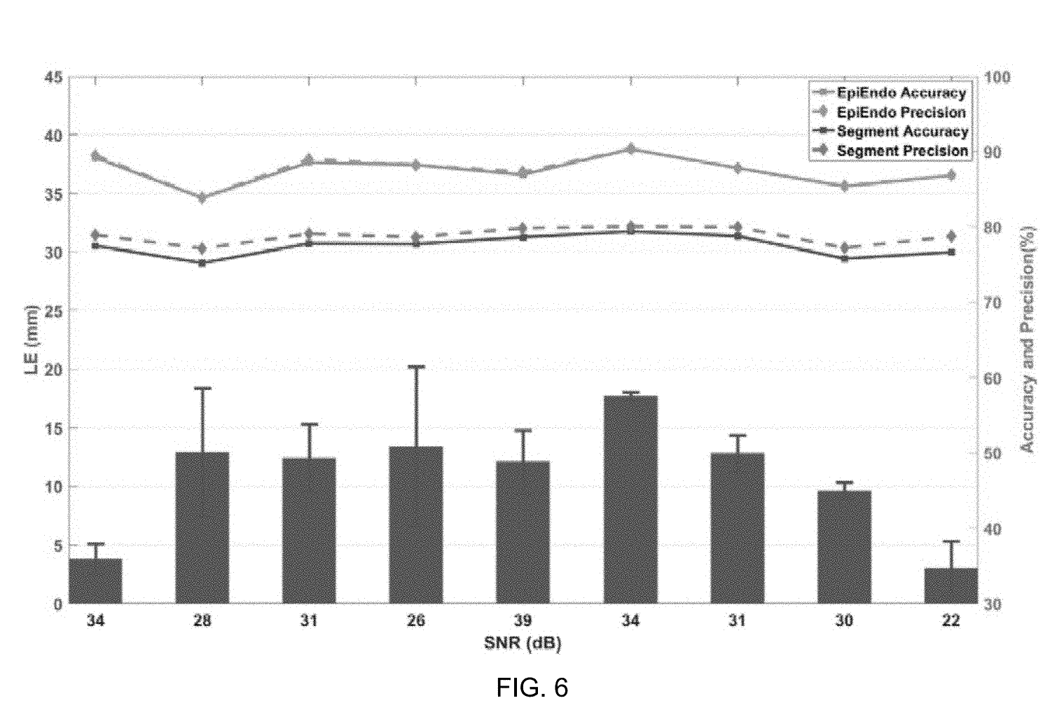

Referring to FIG. 5, an example graph of localization errors (LE) of CNN with heart registration errors is shown. In some configurations, since the ventricle current-dipole model may be registered with a patient's heart and CNNs may be trained and tested using this model and finally use CNNs to localize origins of PVCs, it may be necessary to investigate the influences of heart registration errors on the localization performance of CNNs. FIG. 5 shows the average LE when we trained and tested CNNs with correct registration, and applied to localization when the input 12-lead ECGs were generated by the leadfield with heart registration errors. The SNR of training and testing data is the same as the SNR of input data for testing localization performance. Average registration errors are shown for a variety of registration errors in 20 dB graph 520, 10 dB graph 530, and 5 dB graph 540. Locations include heart move to the right 10 mm 501; heart move to the left 10 mm 502; heart move up 10 mm 503; heart move down 10 mm 504; heart move to the anterior 5 mm 505; heart move to the posterior 5 mm 506; heart rotate right 10° 507; heart rotate left 10° 508; heart rotate upwards 10° 509; heart rotate downwards 10° 510; heart rotate clockwise 10° 511; heart rotate counterclockwise 10° 512; heart inflated 10 mm 513; and heart deflated 10 mm 514.

Three categories of heart registration errors were investigated: shift, rotation and scaling. Overall, all the average LEs are larger than those of correct registration. Within shift group, because heart is already very close to the front body surface, we could only move heart forward by 5 mm. For comparison purpose, we also moved heart backwards by 5 mm. Heart moving to the right always generated a larger LE than moving to the left. Within rotation group, heart rotating to the right always generated a larger LE than rotating to the left. Within scaling group, there is no obvious trend. Among all the registration errors, we found that shift group gave higher LEs than the other two groups on average.

Based on the above analysis of the registration error results, attention may need to be paid when we need to shift the ventricle model for the registration with patient's heart. And CNNs are generally robust to a variety of heart registration errors if we compare these LEs with those that are from correct registrations: 10.43±9.77 mm (20 dB), 10.61±9.64 mm (10 dB) and 11.73±9.80 mm (5 dB). Generally, the above heart registration errors would increase average LE by 1 to 2 mm.

Application to PVC Patients: On average, the localization error of 9 patients is 10.9±5.5 mm, which is good and reasonable considering the source location was estimated solely from 12-lead ECGs. The present results demonstrated the feasibility of employing a generic anisotropic ventricle current-dipole model to mimic cardiac activities in a patient without structural heart disease. Generally, the more similar a patient's heart is with the ventricle model, the more accurate the predictions will be. And this also shows the capability of our method to estimate origins of PVCs from different areas.

Example Applications in Various Embodiments: This method can be applied to the localization of PVC and focal ventricular tachycardia (VT) since the training data is generated from single-site pacing. It is a stable method in the sense of two-fold: once CNNs are trained on a patient, they are applicable to all the other PVCs and focal VTs from the same patient; structures and parameters of both CNNs all remain the same across different subjects and noise levels. It is a robust method because average LEs under different noise levels and heart registration errors fluctuate in a range of 1 to 2 mm. Since a generic ventricle current-dipole model is used, this method does not require high-resolution contrast cardiac CT images. Anatomical CT images show the shape and orientation of ventricles are good enough for the registration purpose. Thus, we reduce patients' burden by avoiding injecting contrast into the body. Training and testing of CNNs could be done off-line prior to the EP study and it generally takes about 20 minutes. The total analysis time including building the boundary element model, registration of the ventricle model, calculating the leadfield between electrodes and ventricle model, and training and testing CNNs would not be longer than 2 hours in a 64-bit operating system with 3.40 GHz Intel i7 processor. Once CNNs have been trained, it would take 0.2 s to calculate the source location given QRS complexes from 12-lead ECGs. Unlike with previous methods—which require calculating statistical features from 12-lead ECGs, require some expertise of recognizing patterns from 12-lead ECGs, and are usually limited in the types of ventricular arrhythmias—the example approach presented here is easy to implement, relieves the burden on electrophysiology specialists, and finally helps guide ablation procedure by providing potential source locations throughout the ventricles.

It is noted that the sources of localization errors mainly come from two places: one is the registration between the ventricle model and patient's heart, the other is the registration of 12-lead electrodes. The latter can be reduced by digitizing more anatomical landmarks to help the registration of electrodes. A personalized cardiac electrophysiology model, as has been used in cardiac electrical imaging studies, can be used to overcome the first source of localization errors.

In various embodiments, the site of origin of premature ventricular contractions can be localized from 12-lead ECG using convolutional neural networks and a realistic anisotropic ventricle computer model. By applying it to real data in a group of 9 PVC patients, we have shown the capability of our method to target the potential ablation site in premature ventricular contractions patients. This new approach for cardiac source localization of origin of arrhythmias using only the 12-lead ECG by means of CNN has important applications for real-time monitoring and localizing origins of cardiac arrhythmias guiding ablation treatment.

In certain implementations, two CNNs (Segment CNN and Epi-Endo CNN) may be used to classify among ventricular sources from 25 segments and from epicardium (Epi) or endocardium (Endo). The inputs are the full time courses and the first half of QRS complexes of 12-lead ECG, respectively. After registering the ventricle computer model with an individual patient's heart, the training datasets were generated by multiplying ventricular current dipoles derived from single pacing at various locations with patient-specific lead field. The origins of PVC are localized by calculating the weighted center of gravity of classification returned by the CNNs. A number of computer simulations were conducted to evaluate the presented approach under a variety of noise levels and heart registration errors. Furthermore, the proposed approach was evaluated on 90 PVC beats from 9 human patients with PVCs and compared against ablation outcome in the same patients.

As disclosed, the computer simulation evaluation returned relatively high accuracies for Segment CNN (˜78%) and Epi-Endo CNN (˜90%). Clinical testing in 9 PVC patients resulted an averaged localization error of 11 mm. The simulation and clinical evaluation results demonstrate the capability and merits of the proposed CNN-based method for localization of PVC. This work suggests a new approach for cardiac source localization of origin of arrhythmias using only the 12-lead ECG by means of CNN, and may have important applications for future real-time monitoring and localizing origins of cardiac arrhythmias guiding ablation treatment.

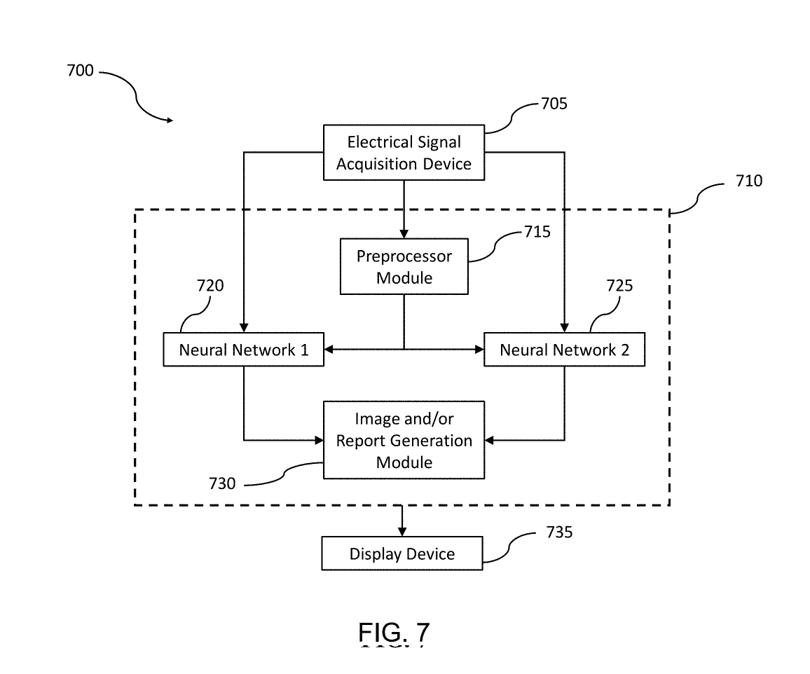

Referring to FIG. 7, an example arrhythmia localization system 700 includes an electrical signal acquisition device 705, a computing device 710, and a display device 735. The electrical signal acquisition device 705 is capable of recording physiological data from the heart. In certain configurations, device 705 is a 12-lead ECG device that is found in most if not all major health clinics. Through selectively-positioned electrodes, device 705 can be used to, for example, sense electrical signals from the heart of a patient and to generate an ECG that includes the QRS complex in the heartbeat cycle. The computing device 710 may include one or more processors and memory for storing instructions that are executed by the one or more processors to provide the functionality being discussed.

The output of device 705 can be fed to a preprocessor module 715, and/or to one or more neural networks 720, 725, depending on the particular format of the output of device 705, and depending on what data is required by the neural network as an input. For example, the preprocessor module 715 may be involved in, for example, registering a generic model of the heart with the patient's heart, selecting/extracting portions of the ECG (such as the first half of the QRS complex), etc. In other implementations, device 705 may output data that can be fed directly to neural networks 720, 725. The outputs of the neural networks 720, 725 may then be provided to an image/report generation module 730. A report generated by module 730 may indicate, for example, a segment of the heart where an arrhythmia originates, or whether the arrhythmia is epicardial or endocardial. An image generated by module 730 may provide a depiction of a heart with a visual marker (such as a circle, star, or other shape) situated to correspond with where in the heart the arrhythmia originates (and consequently, which part of the heart may be a suitable target for ablation by a healthcare provider).

It is noted that the three devices (i.e., devices 705, 710, and 715) represented in FIG. 7 can be integrated into fewer devices (i.e., one or two devices), or can be split into more than 3 devices (i.e., four or more devices). Similarly, the modules (which can be implemented using hardware, software, or a combination thereof) represented in FIG. 7 (and in particular as part of device 710) can be rearranged and reconfigured as deemed suitable. Different devices and modules can be communicatively coupled using wired or wireless communications interfaces. For example, in various alternative arrangements and configurations, the preprocessor module 715 can be integrated with the electrical signal acquisition device 705; the neural networks 720, 725 can be implemented in separate computing devices that are in communication (via wire or wirelessly) with electrical signal acquisition device 705, computing device 710, and/or display device 735; the image and/or report generation module 730 can be integrated with display device 735; etc. It is also noted that in certain implementations, one or more of the devices and/or modules may not be necessary. For example, in certain implementations, only one neural network (such as the segment CNN discussed above) may be used. It is further noted that not all components that may be used in various implementations are necessarily depicted in FIG. 7; that is, other devices and modules not shown may be used. For example, an input device would allow a user to select which outputs (e.g., segment and/or epi-endo) and reports/images are desired; a communications interface may allow the system 700 (or components thereof) to transmit or receive data (such as physiological data or commands/instructions) to and from other devices; etc.

The present invention has been described in terms of one or more preferred embodiments, and it should be appreciated that many equivalents, alternatives, variations, additions, and modifications, aside from those expressly stated, and apart from combining the different features of the foregoing embodiments in varying ways, can be made and are within the scope of the invention. In the above description, a number of specific details, examples, and scenarios are set forth in order to provide a better understanding of the present disclosure. These examples and scenarios are provided for illustration, and are not intended to limit the disclosure in any way. The true scope of the invention will be defined by the claims included in this and any later-filed patent applications in the same family.

Those of ordinary skill in the art, with the included descriptions, will be able to implement appropriate functionality without undue experimentation. References in the specification to an “embodiment,” an “example,” a “version,” an “implementation,” a “configuration,” an “instance,” an “iteration,” etc., indicate that the embodiment, example, version, etc. described may include one or more particular features, structures, or characteristics, but not every embodiment, example, version, etc. necessarily incorporates the particular features, structures, or characteristics. Further, when a particular feature, structure, or characteristic is described in connection with an embodiment, it is believed to be within the knowledge of one skilled in the art to effect such feature, structure, or characteristic in connection with other embodiments whether or not explicitly indicated.

The computerized functionality described above may be implemented in hardware, firmware, software, single integrated devices, multiple devices in wired or wireless communication, or any combination thereof. Computerized functions may be implemented as instructions stored using one or more machine-readable media, which may be read and executed by one or more processors. A machine-readable medium may include any mechanism for storing or transmitting information in a form readable by a machine. For example, a machine-readable medium may include any suitable form of volatile or non-volatile memory. In the drawings, specific arrangements or orderings of schematic elements may be shown for ease of description. However, the specific ordering or arrangement of such elements is not meant to imply that a particular order or sequence of processing, or separation of processes, is required in all embodiments. Further, some connections or relationships between elements may be simplified or not shown in the drawings so as not to obscure the disclosure. This disclosure is to be considered as exemplary and not restrictive in character, and all changes and modifications that come within the spirit of the disclosure are desired to be protected.

Additional background and contextual information and enabling details may be found in the following references, each of which is incorporated by reference in its entirety for all purposes:
E. Corday et al, “Detection of Phantom Arrhythmias and Evanescent Electrocardiographic Abnormalities: Use of Prolonged Direct Electrocardiocording,” JAMA, vol. 193, no. 6, pp. 417-421, August 1965.
R. J. Simpson et al, “Prevalence of premature ventricular contractions in a population of African American and white men and women: the Atherosclerosis Risk in Communities (ARIC) study,” Am. Heart J., vol. 143, no. 3, pp. 535-540, 2002.
L. S. Klein et al, “Radiofrequency catheter ablation of ventricular tachycardia in patients without structural heart disease,” Circulation, vol. 85, no. 5, pp. 1666-1674, May 1992.
M. E. Josephson et al, “Ventricular activation during ventricular endocardial pacing. II. Role of pace-mapping to localize origin of ventricular tachycardia,” Am. J. Cardiol., vol. 50, no. 1, pp. 11-22, July 1982.
R. L. Jadonath et al, “Utility of the 12-lead electrocardiogram in localizing the origin of right ventricular outflow tract tachycardia,” Am. Heart J., vol. 130, no. 5, pp. 1107-1113, 1995.
S. Kamakura et al, “Localization of optimal ablation site of idiopathic ventricular tachycardia from right and left ventricular outflow tract by body surface ECG,” Circulation, vol. 98, no. 15, pp. 1525-1533, 1998.
M. E. Josephson and D. J. Callans, “Using the twelve-lead electrocardiogram to localize the site of origin of ventricular tachycardia,” Heart Rhythm, vol. 2, no. 4, pp. 443-446, 2005.
R. Setiono and H. Liu, “Neural-network feature selector,” IEEE Trans. Neural Netw., vol. 8, no. 3, pp. 654-662, May 1997.
H. A. Rowley et al, “Neural network-based face detection,” IEEE Trans. Pattern Anal. Mach. Intell., vol. 20, no. 1, pp. 23-38, January 1998.
P. D. Heermann and N. Khazenie, “Classification of multispectral remote sensing data using a back-propagation neural network,” IEEE Trans. Geosci. Remote Sens., vol. 30, no. 1, pp. 81-88, January 1992.
G. M. Foody and M. K. Arora, “An evaluation of some factors affecting the accuracy of classification by an artificial neural network,” Int. J. Remote Sens., vol. 18, no. 4, pp. 799-810, March 1997.
H.-S. Chow et al, “Detection of ventricular ectopic beats using neural networks,” in Computers in Cardiology 1992, Proceedings of, 1992, pp. 659-662.
P. de Chazal and R. B. Reilly, “Automatic classification of ECG beats using waveform shape and heart beat interval features,” in Acoustics, Speech, and Signal Processing, 2003. Proceedings. (ICASSP'03). 2003 IEEE International Conference on, 2003, vol. 2, p. 11-269.
S. Osowski and T. H. Linh, “ECG beat recognition using fuzzy hybrid neural network,” IEEE Trans. Biomed. Eng., vol. 48, no. 11, pp. 1265-1271, 2001.
H. Gothwal et al, “Cardiac arrhythmias detection in an ECG beat signal using fast fourier transform and artificial neural network,” J. Biomed. Sci. Eng., vol. 4, no. 04, p. 289, 2011.
K. Minami et al, “Real-time discrimination of ventricular tachyarrhythmia with Fourier-transform neural network,” IEEE Trans. Biomed. Eng., vol. 46, no. 2, pp. 179-185, 1999.
Y. Kaya and H. Pehlivan, “Classification of Premature Ventricular Contraction in ECG,” Int J Adv Comput Sci Appl, vol. 6, no. 7, pp. 34-40, 2015.
N. Allahverdi et al, “Diagnosis of Coronary Artery Disease Using Deep Belief Networks,” in 2. Int. Conf. Eng. Nat. Sci, p. 9.
L. N. Sharma et al, “Multiscale Energy and Eigenspace Approach to Detection and Localization of Myocardial Infarction,” IEEE Trans. Biomed. Eng., vol. 62, no. 7, pp. 1827-1837, July 2015.
Y. Özbay et al, “A fuzzy clustering neural network architecture for classification of ECG arrhythmias,” Comput. Biol. Med., vol. 36, no. 4, pp. 376-388, 2006.
M. Javadi et al, “Improving ECG classification accuracy using an ensemble of neural network modules,” PLoS One, vol. 6, no. 10, p. e24386, 2011.
S. Kiranyaz et al, “Real-Time Patient-Specific ECG Classification by 1-D Convolutional Neural Networks,” IEEE Trans. Biomed. Eng., vol. 63, no. 3, pp. 664-675, March 2016.
B. Hedén et al, “Acute myocardial infarction detected in the 12-lead ECG by artificial neural networks,” Circulation, vol. 96, no. 6, pp. 1798-1802, 1997.
T. Stamkopoulos et al, “ECG analysis using nonlinear PCA neural networks for ischemia detection,” IEEE Trans. Signal Process., vol. 46, no. 11, pp. 3058-3067, 1998.
H. Haraldsson et al, “Detecting acute myocardial infarction in the 12-lead ECG using Hermite expansions and neural networks,” Artif. Intell. Med., vol. 32, no. 2, pp. 127-136, 2004.
M. D. Cerqueira et al, “Standardized myocardial segmentation and nomenclature for tomographic imaging of the heart. A statement for healthcare professionals from the Cardiac Imaging Committee of the Council on Clinical Cardiology of the American Heart Association,” Circulation, vol. 105, no. 4, pp. 539-542, January 2002.
F. Sacher et al, “Epicardial ventricular tachycardia ablation,” J. Am. Coll. Cardiol., vol. 55, no. 21, pp. 2366-2372, 2010.
P. Della Bella et al, “Epicardial Ablation for Ventricular TachycardiaClinical Perspective,” Circ. Arrhythm. Electrophysiol., vol. 4, no. 5, pp. 653-659, 2011.
J. Fernandez-Armenta and A. Berruezo, “How to recognize epicardial origin of ventricular tachycardias?,” Curr. Cardiol. Rev., vol. 10, no. 3, pp. 246-256, 2014.
A. Berruezo et al, “Electrocardiographic recognition of the epicardial origin of ventricular tachycardias,” Circulation, vol. 109, no. 15, pp. 1842-1847, 2004.
P. M. van Dam et al, “Application of the fastest route algorithm in the interactive simulation of the effect of local ischemia on the ECG,” Med. Biol. Eng. Comput., vol. 47, no. 1, pp. 11-20, January 2009.
P. M. van Dam et al, “Quantitative localization of premature ventricular contractions using myocardial activation ECGI from the standard 12-lead electrocardiogram,” J. Electrocardiol., vol. 46, no. 6, pp. 574-579, 2013.
D. Potyagaylo et al, “Influence of Modeling Errors on the Initial Estimate for Nonlinear Myocardial Activation Times Imaging Calculated with Fastest Route Algorithm,” IEEE Trans. Biomed. Eng., vol. 63, no. 12, pp. 2576-2584, December 2016.
R. Cabrera-Lozoya et al, “Image-Based Biophysical Simulation of Intracardiac Abnormal Ventricular Electrograms,” IEEE Trans. Biomed. Eng., vol. 64, no. 7, pp. 1446-1454, July 2017.
Z. Liu et al, “Noninvasive reconstruction of three-dimensional ventricular activation sequence from the inverse solution of distributed equivalent current density,” IEEE Trans. Med. Imaging, vol. 25, no. 10, pp. 1307-1318, 2006.
L. Yu et al, “Temporal sparse promoting three dimensional imaging of cardiac activation,” IEEE Trans. Med. Imaging, vol. 34, no. 11, pp. 2309-2319, 2015.
C. Han et al, “Noninvasive imaging of three-dimensional cardiac activation sequence during pacing and ventricular tachycardia,” Heart Rhythm, vol. 8, no. 8, pp. 1266-1272, 2011.
C. Han et al, “Noninvasive cardiac activation imaging of ventricular arrhythmias during drug-induced QT prolongation in the rabbit heart,” Heart Rhythm, vol. 10, no. 10, pp. 1509-1515, 2013.
C. Han et al, “Noninvasive reconstruction of the three-dimensional ventricular activation sequence during pacing and ventricular tachycardia in the canine heart,” Am. J. Physiol.-Heart Circ. Physiol., vol. 302, no. 1, pp. H244-H252, 2012.
C. Han et al, “Imaging cardiac activation sequence during ventricular tachycardia in a canine model of nonischemic heart failure,” Am. J. Physiol.-Heart Circ. Physiol., vol. 308, no. 2, pp. H108-H114, 2015.
C. Liu et al, “Noninvasive Mapping of Transmural Potentials During Activation in Swine Hearts From Body Surface Electrocardiograms,” IEEE Trans. Med. Imaging, vol. 31, no. 9, pp. 1777-1785, September 2012.
Z. Zhou et al, “Noninvasive Imaging of 3-Dimensional Myocardial Infarction from the Inverse Solution of Equivalent Current Density in Pathological Hearts,” IEEE Trans. Biomed. Eng., vol. 62, no. 2, pp. 468-476, February 2015.
Z. Zhou et al, “Noninvasive Imaging of High-Frequency Drivers and Reconstruction of Global Dominant Frequency Maps in Patients with Paroxysmal and Persistent Atrial Fibrillation,” IEEE Trans. Biomed. Eng., vol. 63, no. 6, pp. 1333-1340, 2016.
B. He et al, “Noninvasive imaging of cardiac transmembrane potentials within three-dimensional myocardium by means of a realistic geometry anisotropic heart model,” IEEE Trans. Biomed. Eng., vol. 50, no. 10, pp. 1190-1202, 2003.
“Deep Learning Toolbox—File Exchange—MATLAB Central.” [Online]. Available: http://www.mathworks.com/matlabcentral/fileexchange/38310-deep-learning-toolbox. [Accessed: 11 Aug. 2017].
F. N. Wilson et al, “Electrocardiograms that represent the potential variations of a single electrode,” Am. Heart J., vol. 9, no. 4, pp. 447-458, April 1934.
S. Giffard-Roisin et al, “Non-Invasive Personalisation of a Cardiac Electrophysiology Model from Body Surface Potential Mapping,” IEEE Trans. Biomed. Eng., vol. PP, no. 99, pp. 1-1, 2017.

What is claimed is:

1. A method for identifying an arrhythmia origination location in a heart of a subject, the method comprising:
acquiring electrical data recorded using an electrocardiography device, the electrical data corresponding to electrical activity in the heart of the subject;
providing the electrical data to one or more neural networks; and
receiving from the one or more neural networks an identification of a segment of the heart at which an arrhythmia originates.

2. The method of claim 1, wherein the identification of the segment comprises generating and displaying an image visually depicting the segment of the heart, relative to other segments, in which the arrhythmia originates.

3. The method of claim 1, wherein the identification of the segment of the heart at which the arrhythmia originates is based on physiological data consisting of the electrical data.

4. The method of claim 1, wherein the electrocardiography device comprises a 12-lead ECG, wherein the one or more neural networks comprises one or more convolutional neural networks, and wherein the identification of the segment of the heart at which the arrhythmia originates is based on providing physiological data consisting of the electrical data from the 12-lead ECG to the one or more convolutional neural networks.

5. The method of claim 1, further comprising building a personalized leadfield based on at least one of a CT image or an MRI image of a torso and the heart of the subject.

6. The method of claim 1, wherein the one or more neural networks comprises one or more convolutional neural networks (CNNs), and wherein the one or more CNNs comprises a segment CNN that receives as input full time courses of an electrocardiogram and provides as output the segment of the heart at which the arrhythmia originates.

7. The method of claim 1, wherein the one or more neural networks comprises one or more convolutional neural networks (CNNs), and wherein the one or more CNNs comprises an epi-endo CNN that receives as input a portion of a QRS complex acquired from an electrocardiogram and provides as output an identification of whether the arrhythmia has an epicardial focus or an endocardial focus.

8. The method of claim 1, wherein the one or more neural networks comprises:
a segment convolutional neural network (CNN) to provide the identification of the segment; and
an epi-endo CNN to identify whether the arrhythmia has an epicardial focus or an endocardial focus.

9. The method of claim 8, wherein the segment CNN receives as input a full time course of an electrocardiogram, and the epi-endo CNN receives as input a portion of a QRS complex from the electrocardiogram.

10. The method of claim 1, further comprising treating the segment of the heart at which the arrhythmia originates using ablation.

11. A system for identifying an arrhythmia origination location in a heart of a subject, the system comprising:
an electrocardiography device to record electrical data corresponding to electrical activity in the heart;
a processor and memory having instructions that, when executed by the processor, are to:
use the electrocardiography device to record electrical activity in the heart of the subject,
provide the electrical data to one or more neural networks, and
receive from the one or more neural networks an identification of a segment of the heart at which an arrhythmia originates.

12. The system of claim 11, wherein the identification of the segment comprises generating and displaying an image visually depicting the segment of the heart, relative to other segments, in which the arrhythmia originates.

13. The system of claim 11, wherein the identification of the segment of the heart at which the arrhythmia originates is based on physiological data consisting of the electrical data.

14. The system of claim 11, wherein the electrocardiography device comprises a 12-lead ECG, wherein the one or more neural networks comprises one or more convolutional neural networks, and wherein the identification of the segment of the heart at which the arrhythmia originates is based on providing physiological data consisting of the electrical data from the 12-lead ECG to the one or more convolutional neural networks.

15. The system of claim 11, the memory further comprising instructions to build a personalized leadfield based on at least one of a CT image or an MRI image of a torso and the heart of the subject.

16. The system of claim 11, wherein the one or more neural networks comprises one or more convolutional neural networks (CNNs), and wherein the one or more CNNs comprises a segment CNN that receives as input full time courses of an electrocardiogram and provides as output the segment of the heart at which the arrhythmia originates.

17. The system of claim 11, wherein the one or more neural networks comprises one or more convolutional neural networks (CNNs), and wherein the one or more CNNs comprises an epi-endo CNN that receives as input a portion of a QRS complex acquired from an electrocardiogram and provides as output an identification of whether the arrhythmia has an epicardial focus or an endocardial focus.

18. The system of claim 11, wherein the one or more neural networks comprises:
a segment convolutional neural network (CNN) to provide the identification of the segment; and
an epi-endo CNN to identify whether the arrhythmia has an epicardial focus or an endocardial focus.

19. The system of claim 18, wherein the segment CNN receives as input a full time course of an electrocardiogram, and the epi-endo CNN receives as input a portion of a QRS complex from the electrocardiogram.

20. The system of claim 11, further comprising an ablation device, wherein the memory further comprising instructions to use the ablation device to ablate the segment of the heart at which the arrhythmia originates.
